(19) United States
(12) Patent Application Publication
(10) Pub. No.: US 2020/0231822 A1
(43) Pub. Date: Jul. 23, 2020

(54) ELECTRICALLY CONDUCTIVE ANTIFOULING COATING COMPOSITION

(71) Applicant: PRESIDENT AND FELLOWS OF HARVARD COLLEGE, Cambridge, MA (US)

(72) Inventors: Pawan JOLLY, Boston, MA (US); Olivier Yves Frederic HENRY, Brookline, MA (US); Donald E. INGBER, Boston, MA (US); Jonathan SABATE DEL RIO, Tarragona (ES)

(73) Assignee: PRESIDENT AND FELLOWS OF HARVARD COLLEGE, Cambridge, MA (US)

(21) Appl. No.: 16/617,566

(22) PCT Filed: Jul. 27, 2018
(86) PCT No.: PCT/US2018/044076

§ 371 Date: Nov. 27, 2019

Related U.S. Application Data
(60) Provisional application No. 62/537,829, filed on Jul. 27, 2017.

(51) Int. Cl. C09D 5/24; C09D 7/61; C09D 7/62; C09D 189/00; G01N 33/543

(52) U.S. Cl. CPC C09D 5/24; C09D 7/61; C08K 2003/0831; C09D 189/00; G01N 33/5438; C09D 7/62

(57) ABSTRACT

Carbon nanotubes or graphene combined with proteinaceous material forming compositions that can be coated on surfaces are described. For example, the described compositions can be used as a coating on an electrode. The coatings can be functionalized with capture agents, targeting specific analytes. In addition to being conductive, the coatings prevent fouling and passivation of the electrodes by non-specific binding. This allows the coated electrodes to be used in complex matrices such as can be found in biological fluids and tissues. The coated electrodes can be regenerated and reused repeatedly.

### RELATED APPLICATIONS

This application claims benefit under 35 U.S.C. § 119(e) of the U.S. Provisional Application No. 62/537,829 filed Jul. 27, 2017, the content of which is incorporated herein by reference in its entirety.

### GOVERNMENT SUPPORT

This invention was made with government support under contract W911NF-12-2-0036 awarded by the Department of Defense. The government has certain rights in the invention.

### FIELD OF THE INVENTION

This invention relates to compositions and methods for making electrically conducting coatings and their use. For example, coatings for electrodes that prevent non-specific binding and fouling of the electrode surface.

### BACKGROUND

Molecular diagnostics and assays rely on the specific interaction between a capture agent and a target of interest. While selectivity is an inherent property of the capture agent's affinity for its target, non-specific interactions can considerably decrease the sensitivity of an assay and result in false positives.

Molecular blockers of varying molecular weight, including Bovine Serum Albumin (BSA), casein, pluronic acid, and poly(ethylene glycol) polymers (PEG) among others, have been used to limit non-specific binding interactions that may occur at surfaces and/or in solution. For example, the surface of microtiter plates used in enzyme linked sandwich immunoassays (ELISAs) are typically blocked with BSA to reduce non-specific adsorption of proteins at their surface and BSA is also usually added to the buffers used during the assay.

For assays based on a final optical readout (e.g. absorbance, fluorescence, chemiluminescence or electrochemiluminescence), the blockers do not interfere with the final measurement. This is because the assay chemistry and measurements are fully decoupled. The assay is carried out on a surface (e.g. plates, microbeads and nanoparticles) whereas the final measurement is performed using an external transducer. For example, in fluorescence based assays, light of a predetermined wavelength is shined on a surface bearing the capture agent and the light emitted is quantified by a photodiode or CCD sensor (i.e., the transducer). In the forgoing example, the surface where the molecular interaction takes place acts as a passive support and does not contribute to the measurement.

A more challenging situation is presented when an electrochemical read out is desired since the assay is carried out on the transducer surface. The capture agent is typically immobilized at the surface of an electrode using strategies that should maximize its density and orientation, prevent non-specific interactions, and at the same time, preserve the ability of the electrode to record electrochemical signals with high sensitivity. Molecular blockers have been used to prevent non-specific interactions, but often result in passivation of the electrodes and consequently lead to a dramatic loss in sensitivity. Existing use of electrochemical sensors therefore involves a constant trade-off between sensitivity and blocking that requires difficult optimization.

Finally, complex samples containing proteins and/or biofouling agents in large concentrations (e.g. blood, plasma) cannot be analyzed without prior dilutions as they will further block the electrode surface which rapidly results in the complete and irreversible passivation of the electrochemical sensor. Importantly, this is a major limitation that must be circumvented for all biosensors (not just electrochemical sensors).

U.S. Pat. No. 8,778,269 describes the fabrication of nanoelectronic electrochemical test devices for detection of biomolecules electrochemically in a variety of ways. This patent does not describe a robust denatured and cross-linked composite as the conductive coating and the use of the preparations as an antifouling nanocomposite are also not described.

There is therefore a need for coatings that can be used on electrically conductive surfaces that can accommodate capture agents, prevent non-specific interaction and preserve the ability of the electrode to record electrochemical signals with high sensitivity. The present disclosure addresses some of these needs.

### SUMMARY

In general, the inventions described herein relate to compositions that can be applied to conducting surfaces and protect these surfaces from unwanted interactions that impede or diminish their intended function. For example, the coatings can be applied to electrodes, providing an electrode that can be utilized in complex matrices such as blood and plasma. Furthermore, some embodiments described herein allow for electrochemical measurements in complex matrices without complicated purification and dilution steps. In addition, the coatings herein described can be sterilized, are easy to functionalize, are robust and are easy to prepare.

In one aspect the invention comprises a mixture of an allotrope of carbon having atoms arranged in a hexagonal lattice and a proteinaceous material, wherein the proteinaceous material is non-reversibly denatured. For example, the allotrope can be carbon nanotubes or graphene, or a functionalized material such as carboxylated carbon nanotubes (herein referred to as CNTs or CNT), aminated carbon nanotubes, reduced graphene oxide (rGO), carboxylated reduced graphene oxide (RG-Carboxylate), aminated reduced graphene oxide (RG-Amino), and mixtures of these. Optionally, the proteinaceous material can be BSA and optionally, the proteinaceous material is cross-linked. The composition can also further comprise a capture agent and/or a conductive surface (e.g., an electrode surface).

In another aspect, the invention is for an electrode. The electrode comprises a conductive surface, such as a metal or glassy carbon. The electrode further comprises a mixture of an allotrope of carbon having atoms arranged in a hexagonal lattice and a non-reversibly denatured proteinaceous material coated on at least a part of the conductive surface. The proteinaceous material can be cross-linked. Optionally, the mixture conducts vertically to a greater degree than laterally, for example when coated on the electrode. The electrode can also optionally be multiplexed.

In yet another aspect, the invention if for a method of making an electrode coating composition. The method comprises mixing an allotrope of carbon having carbon atoms arranged in a hexagonal lattice (e.g., carboxylated nanotubes, reduced graphene oxide) and proteinaceous material in a solution (e.g., an aqueous solution). Furthermore, the proteinaceous material is non-reversibly denatured prior to or after mixing with the allotrope of carbon. Optionally the method includes sonicating the allotrope of carbon and proteinaceous mixture. Also, optionally the proteinaceous material is heated, for example to denature the material. The method can also include cross-linking the proteinaceous material. Optionally the method includes purifying the allotrope of carbon and proteinaceous mixture.

Finally, an aspect of the invention includes a method of making a coated electrode. The method comprises coating at least a portion of a conducting surface with a mixture of an allotrope of carbon having carbon atoms arranged in a hexagonal lattice (e.g., CNTs, reduced graphene oxide) and a proteinaceous material, wherein the proteinaceous material is non-reversibly denatured. Optionally the method further comprises cross-linking the proteinaceous material. The mixture can include a capture agent. Optionally, the electrode is coated with the carbon allotrope/proteinaceous material and then functionalized, for example, with a capture agent.

In addition to accommodating capture agents, preventing non-specific interaction and fouling of electrode, and preserving the ability of electrodes to record electrochemical signals with high sensitivity, the inventions described herein have other useful properties and applications. It has been discovered, for example, that the coatings can be made with a highly anisotropic electrical conductivity. This anisotropy can be exploited to make electrodes which conduct vertically but not laterally (e.g., with respect to the electrode surface) allowing arrays of adjacent electrodes to be coated with an overlapping coating that can span one or more electrode since the coating will not conduct between the electrodes. This makes the coatings easy to apply, and can protect the entire surface (e.g., electrode and insulator between electrodes) since a larger area covering several electrodes can be coated rather than careful application to individual electrodes to avoid electrical contact if the coating were conductive laterally. The coated electrodes described herein also can be used where long term passive electrical and electrochemical recordings in whole tissues have previously been challenging. For example, for neuronal recordings. Other applications include implantable stimulation or recording electrodes or biosensors. In some embodiments the coatings are transparent and can therefore find application in solar cell technologies and as coatings for transparent conductors such as ITO. The coatings are also robust and can be cleaned and reused repeatedly with little or no loss of sensor sensitivity.

### BRIEF DESCRIPTION OF THE FIGURES

FIG. 1 is a highly diagrammatic depiction of a gold electrode that has been coated with a BSA/CNTs composition, (e.g., “e.Block”) and functionalized with a capture antibody (Capture Ab) through an amide linkage. The figure also shows captured antigen IL6 that is detected with a biotinylated detection antibody conjugated to streptavidin-polyHRP. Sacrificial redox active agent 3,3′,5,5′-Tetramethylbenzidine (TMB) is shown (top) as being oxidized (middle) and precipitated (bottom, proximate to BSA/CNTs) onto the electrode surface where it can be detected electrochemically (e.g., by reduction, or reduction and oxidation cycles such as used in cyclic voltammetry).

FIG. 2 is a graph showing the electrochemical signal from the oxidation current density (bars) and peak to peak voltage difference (filled circle markers) of a 5 mM ferri/ferrocyanide in phosphate buffer saline solution (PBS) for a series of electrodes. From left to right: bare gold electrode; gold+1% BSA after 30 min; a self-assembled monolayer of a PEGylated thiol (SAM) functionalized gold electrode; the SAM functionalized electrode+1% BSA after 30 min; an e.Block coated gold electrode; e.Block+1% BSA after 30 min; e.Block+1% BSA after 1 week; e.Block+1% BSA after 1 month.

FIG. 3 shows UV spectrum of materials that can be used for coating electrodes. Single walled carbon nanotubes (SWCNT) and SWCNT denatured show almost no adsorption in the scanned region. BSA, BSA denatured, and comparative sample PTNTM show pronounced adsorption peaks at 230 nm and 280 nm. BSA/CNTs denatured series shows significant reduction at the 230 nm and 280 nm bands.

FIG. 4 is a fluorescence image of an array of 6 gold sensors. The image shows, from top to bottom, an unmodified gold sensor, a gold sensor incubated with e.Block+2.5% glutaraldehyde for 24 hours and a sensor treated with (1-ethyl-3-(3-dimethylaminopropyl)carbodiimide hydrochloride)/Dicylohexylcarbodiimide (EDC/NHS). The sensors were spotted with green fluorescent protein (GFP) or PBS.

FIG. 5 shows a plot of the relative fluorescent pixel intensity on the surface of gold sensors treated with GFP, e.Block+2.5% glutaraldehyde (GA), and e.Block+2.5% GA+EDC/NHS.

FIG. 6 shows a plot of oxidation current density (bars) and peak to peak voltage difference (filled circle markers) of a 5 mM ferri/ferrocyanide in PBS for untreated and treated gold electrode sensors that have been immersed in a BSA containing solution. Untreated gold is passivated within 30 min, e.Block treated shows a high current density that shows no change in current density over four days, while a comparative treatment shows no significant current density at two time points.

FIG. 7 is a plot showing the electrochemical signal from the oxidation current density (bars) and peak to peak voltage difference (filled circle markers) of a 5 mM ferri/ferrocyanide in PBS buffer solution following O₂ plasma sterilization. Measurements are, from left to right, bare gold electrodes, electrodes modified with e.Block and treated with a O₂ plasma (0.3 mbar, 50 watt, 4 minutes), and the e.Block coated and O₂ treated electrode signal after incubation in 1% BSA for 1 month.

FIG. 8 is a plot showing the electrochemical signals from the oxidation of precipitating TMB recorded for varying concentration of IL6 in the presence of 1% BSA.

FIG. 9 is a diagrammatic depiction showing the performance of a chip functionalized with capture anti-IL6, stored for a week in 1% BSA containing solution, and then used to carry out the detection of 200 pg/mL of IL6 in a solution containing 1% BSA.

FIG. 10 is a voltammogram showing the redox peaks of precipitated TMB after an IL6 detection assay.

FIG. 11 is a voltammogram of an electrode that has been regenerated using HCl·Glycine (HCL·Gly).

FIG. 12 is a voltammogram showing the redox peaks of precipitated TMB in PBS after an IL6 detection assay using a regenerated electrode.

FIG. 13 is a bar graph showing the Faradaic oxidation peak current recorded in redox solution on aminated reduced graphene (RG-Amino)/BSA and carboxylated reduced graphene (RG-Carboxylate)/BSA coatings challenged against undiluted human plasma.

### DETAILED DESCRIPTION

The methods, compositions and structures provided herein are based in part on the use of carbon nanotubes and reduced graphene oxide mixtures with proteinaceous materials to form a conductive and protective coating when applied to surfaces. This invention allows for the formation of an electrochemically active surface blocker that can prevent non-specific interaction when applied to an electrode surface. In some examples, the proteinaceous material is denatured and cross-linked, forming a robust surface that can be reconditioned and re-used repeatedly in complex matrix materials such as blood and serum.

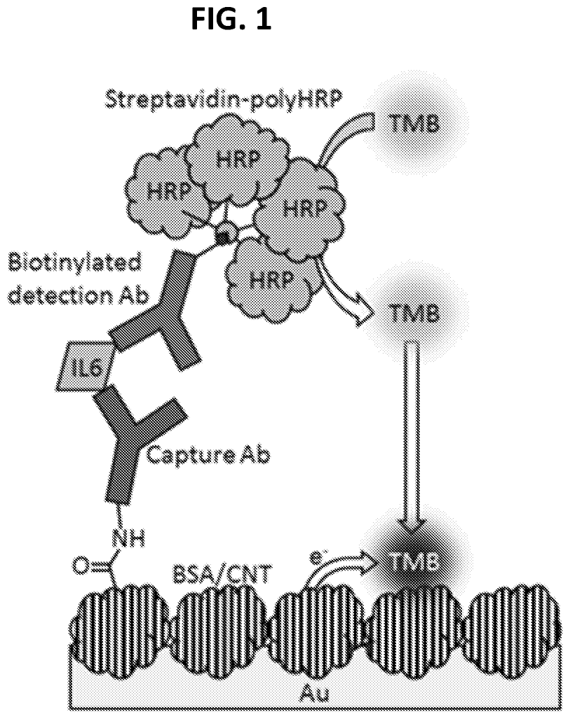

In some of the embodiments the invention includes an electrochemically active surface blocker that can prevent non-specific interaction while keeping the electrode surface active, referred to herein as “e.Blocker” or “e.Block.” The e.Blocker is composed of carbon allotrope (e.g., carbon nanotubes, graphene and/or reduced graphene oxide) mixed with denatured BSA to form a BSA/CNTs nanocomposite that is coated on the electrode surface. FIG. 1 shows an embodiment of the invention. The figure shows a gold electrode that has been coated with e.Blocker, made with CNTs, and functionalized with a capture antibody. The captured antigen IL6 is detected with a biotinylated detection antibody conjugated to streptavidin-polyHRP. TMB is depicted as being oxidized, precipitated onto the electrode surface where it can be detected electrochemically (e.g., by reduction, or reduction and oxidation cycles such as used in cyclic voltammetry). In some embodiments, the nanocomposite e.Blocker can be used to either (i) block an electrode already modified with a capture agent, or in some embodiments (ii) coat a clean electrode and later be modified with the capture agent. FIG. 1 is illustrative and in different embodiments of the other capture agents and other antigens or target can be used.

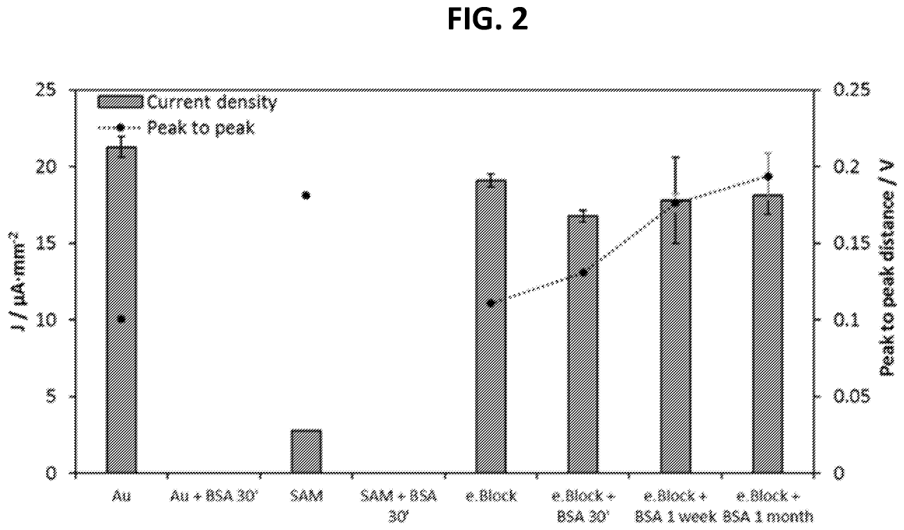

FIG. 2 shows a result of coating a clean electrode with a composition as described herein. As shown in FIG. 2, a bare gold electrode immersed in 1% BSA only needs 30 minutes to lose its ability to respond to the electrochemical tracer ferri/ferrocyanide present in solution. The sensitivity of the gold sensors after applying the e.Block, made here with CNTs, is preserved, dropping by only 10%. In comparison, SAM coated electrodes lost over 80% of their initial sensitivity. In addition, electrodes coated with e.Block retained 85% activity after exposure to 1% BSA for over 1 month. Bare electrodes and SAM coated electrodes were insulated after only 30 minutes. exposure.

As used herein, a “capture agent” is a natural or synthetic receptor (e.g., a molecular receptor) that binds to a target molecule. In some embodiments the binding is a specific binding such that it is selective to that target above non-targets. For example the dissociation constant between the capture agent and target is at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain embodiments, the specific binding refers to binding where the capture agent binds to its target without substantially binding to any other species in the sample/test solution.

By way of non-limiting examples, a capture agent can be an antibody, adnectins, ankyrins, other antibody mimetics and other protein scaffolds, aptamers, nucleic acid (e.g., an RNA or DNA aptamer), protein, peptide, binding partner, oligosaccharides, polysaccharides, lipopolysaccharides, cellular metabolites, cells, viruses, subcellular particles, haptens, pharmacologically active substances, alkaloids, steroids, vitamins, amino acids, avimers, peptidomimetics, hormone receptors, cytokine receptors, synthetic receptors, sugars or molecularly imprinted polymer. The capture agent is selective to a specific target or class of targets such as toxins and biomolecules. For example, the target can be ions, molecules, oligomers, polymers, proteins, peptides, nucleic acids, toxins, biological threat agents such as spore, viral, cellular and protein toxins, carbohydrates (e.g., mono saccharides, disaccharides, oligosaccharides, polyols, and polysaccharides) and combinations of these (e.g., copolymers including these).

In some embodiments the capture agent is an antibody. As used herein, the terms “antibody” and “antibodies” include polyclonal antibodies, monoclonal antibodies, humanized or chimeric antibodies, single chain Fv antibody fragments, Fab fragments, and F(ab)2 fragments. Antibodies having specific binding affinity for a target of interest (e.g., an antigen) can be produced through standard methods. As used herein, the terms “antibody” and “antibodies” refer to intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

In some embodiments the target of the capture agent can be redox active (e.g., an electroactive capture agent) and is directly detected by the electrode. For example, the capture agent facilitates detection of the target analyte by the electrode due to it concentrating the analyte near or at the surface of the electrode where it can be detected directly by electrochemical means. In some other embodiments the target is detected indirectly by electrochemical means. For example, the target can be detected by binding with a detection antibody, protein or molecule that catalyzes, directly or indirectly, a redox reaction close to an electrode surface. Optionally, the detection antibody, protein or molecule deposits a sacrificial redox active molecule on the electrode surface (e.g., on a coating that is on the metal surface of the electrode) that then is detected electrochemically. For example, the detection antibody can be conjugated with a redox catalyst and the sacrificial redox active molecule can be oxidized or reduced and precipitated onto the electrode surface. In some embodiments the redox active catalyst is a peroxidase such as horseradish peroxidase (HRP) and the sacrificial redox active molecule is 3,3′-Diaminobenzidine (DMB); 2,2′-azino-bis(3-ethylbenzothiazoline-6-sulphonic acid) (ABTS); o-orthophenylenediamine (OPD); AmplexRed; 3,3′-Diaminobenzidine (DAB); 4-chloro-1-naphthol (4CN); AEC; 3,3′,5,5′-Tetramethylbenzidine (TMB); homovanilllic acid; lumininol; Nitro blue tetrazolium (NBT); Hydroquinone; benzoquinone; mixtures of these; or mixtures of these. Embodiments include known immunoassays or modifications of these to be detectable by electrochemistry. Optionally, the sacrificial molecule can also be detected by fluorescence.

As used here a “conductive surface” is an outer surface of a bulk conductive material. For example, any surface of a metal sheet, bar, wire, electrode, contact, etc. This can include porous materials, polished materials or materials with any surface roughness, surfaces that are substantially flat or have some curvature (e.g., concave or convex). Conductive surfaces include surfaces of non-metallic materials that are poor conductors or good conductors, such as, for example graphite, Indium tin oxide (ITO), semiconductors, conductive polymers and materials used for making electrodes. For example, the conductivity can be in the range between a semiconductor (e.g., about 1×10³ S/m) and a metal (e.g., about 5×10⁷ S/m) In some embodiments the conductive surface is the part of an electrode that is coated with a protective coating such as a e.Blocker, CNTs/BSA or rGO/BSA compositions, and then contact with the sample that is being probed for an electrochemical response.

As used here a “complex matrix” can include biomolecules, molecules, ions, cells, organisms, inorganic materials, liquids and tissue. For example, a complex matrix can include biological fluids; such as blood, serum, plasma, urine, saliva, interstitial fluid and cytosol; and tissues such as from a biopsy and tissues on a living organism (e.g., an implant, a diagnostic probe).

As used herein a “blocking agent” or “molecular blockers” are compounds used to prevent non-specific interactions. The blocking agent can be a coating on a surface that prevents non-specific interactions or fouling of the surface when it is contacted or immersed in a complex matrix. The surface can include a capture agent, for example, directly attached to the surface or attached to the blocking agent. Non-specific interactions can include any interaction that is not desired between the target molecule and the surface, or between other components in solution. The blocking agent can be a protein, mixture of proteins, fragments of proteins, peptides or other compounds that can passively absorb to the surface in need of blocking. For example proteins (e.g., BSA and Casein), poloxamers (e.g., pluronics), PEG-based polymers and oligomers (e.g., diethylene glycol dimethyl ether), cationic surfactants (e.g., DOTAP, DOPE, DOTMA). Some other examples include commercially available blocking agent or components therein that are available from, for example, Rockland Inc. (Limeric, Pa.) such as: BBS Fish Gel Concentrate; PBS Fish Gel Concentrate; TBS Fish Gel Concentrate; Blocking Buffer for Fluorescent Western Blotting; BLOTTO; Bovine Serum Albumin (BSA); ELISA Microwell Blocking Buffer; Goat Serum; IPTG (isopropyl beta-D-thiogalactoside) Inducer; Normal Goat Serum (NGS); Normal Rabbit Serum; Normal Rat Serum; Normal Horse Serum; Normal Sheep Serum; Nitrophenyl phosphate buffer (NPP); and Revitablot™ Western Blot Stripping Buffer.

As used herein an “electrode” is a conductor through which current enters or leaves a medium, where the medium is nonmetallic. For example, the medium can be a complex matrix (e.g., blood or serum). The electrode can be inserted into/onto a tissue such as mammalian tissue and be contacted with tissue and/or fluids therein/thereon. The electrode can be large (e.g., with a working surface area of greater than 1 cm², greater than 10 cm², greater than 100 cm²) or the electrode can be small (e.g., with a working surface area of less than 1 cm², less than 1 mm², less than 100 μm², less than 10 μm², less than 1 μm²). The working surface area is the area in contact with the medium and wherein current enters or leaves the medium. In some embodiments the electrode is a working electrode and the electrochemical cell can include a counter electrode and reference electrode.

In some embodiments the electrode is “Multiplexed” such that it is configured for a multiplexed assay. As used herein a “multiplexed” assay can be used to simultaneously measure multiple analytes or signals such as two or more (e.g., 3 or more, 5 or more, 10 or more, 50 or more, 100 or more, 1000 or more) during a single run or cycle of the assay. The electrode can therefore be configured as an array of electrodes, microelectrodes or electrochemical sensors each of which can be independently electrically attached to a circuit for monitoring the electrical signals. For example, the array of electrodes can be disposed at the bottom, sides or top of a multiwell plate (e.g., microwell plate) arrayed on a flat surface such as a semiconductor chip (e.g., a sensor array chip) or form part of a multielectrode array (e.g., for connection of neurons to electronic circuitry). In some embodiments, the coatings as described herein e.g., e.Block, can coat more than one sensor since the coating will not conduct between the sensors due to the anisotropy of the conduction, therefore an array of conductors, sensors or electrodes can be coated forming a multiplexed electrode.

Electrodes can include materials with metallic conduction and semiconductors. For example, electrodes can include metals, metal alloys, semiconductors, doped materials, conducting ceramics and conducting polymers. Without limitation, electrode materials can include carbon (e.g., graphite, glassy carbon, conductive polymers), copper, titanium, brass, mercury, silver, platinum, palladium, gold, rhodium, zinc, lead, tin, iron, Indium Tin Oxide (ITO), silicon, doped silicon, II-VI semiconductors (e.g., ZnO, ZnS, CdSe), III-V semiconductors such as (e,g., GaAs, InSb), ceramics (e.g., TiO₂, Fe₃O₄, MgCr₂O₄), and conductive polymers (e.g., poly(acetylene)s, poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, and poly(3,4-ethylenedioxythiophene)), combinations, mixtures and alloys of these. In some embodiments the electrode includes CNTs and CNTs, such as a mixture of CNTs and a proteinaceous material coated on at least a part of a conductive surface comprising the materials listed above. In some embodiments, the electrode can be an electrochemical sensor. Electrodes can also include insulating components such as insulators for electrical and mechanical protection, imparting rigidity and electrical isolation to parts of the electrode.

Electrochemical methods are methods that rely on a change in the potential, charge or current to characterize the analyte's chemical reactivity. Some examples include potentiometry, controlled current coulometry, controlled-potential coulometry, amperometry, stripping voltammetry, hydrodynamic voltammetry, polarography, stationary electrode voltammetry, pulsed polarography, electrochemical impedance spectroscopy and cyclic voltammetry. The signals are detected using an electrode or electrochemical sensors coupled to circuits and systems for collection, manipulation and analysis of the signals.

As used herein “proteinaceous” material includes proteins and peptides, functionalized proteins, copolymers including proteins, natural and synthetic variants of these, and mixtures of these. For example, proteinaceous material can be Bovine Serum Albumin (BSA).

As used herein, “to cross link” means to form one or more bonds between polymer chains so as to form a network structure such as a gel or hydrogel. The polymers are then “cross-linked” polymers. The bonding can be through hydrogen bonding, covalent bonding or electrostatic. The “cross linking agent” can be a bridging molecule or ion, or it can be a reactive species such as an acid, a base or a radical producing agent.

For molecular cross linking agents, the cross linking agents contain at least two reactive groups that are reactive towards numerous groups, including primary amines, carboxyls, sulfhydryls, carbohydrates and carboxylic acids. Proteins and peptide molecules have many of these functional groups and therefore proteins and peptides can be readily conjugated and cross-linked using these cross linking agents. Cross linking agents can be homobifunctional, having two reactive ends that are identical, or heterobifunctional, having two different reactive ends. In some embodiments the cross linking agent is a molecule such as glutaraldehyde, dimethyl adipimidate (DMA), dimethyl suberimidate (DMS), Bissulfosuccinimidyl suberate, formaldehyde, p-azidobenzoyl hydrazide; n-5-azido-2-nitrobenzoyloxysuccinimide; n-[4-(p-azidosalicylamido)butyl]-3′-(2′-pyridyldithio) propionamide; p-azidophenyl glyoxal monohydrate; bis [b-(4-azidosalicylamido)ethyl]disulfide; bis [2-(succinimidooxycarbonyloxy)ethyl] sulfone; 1,4-di [3′-(2′-pyridyldithio)propionamido] butane; dithiobis(succinimidyl propionate); disuccinimidyl suberate; disuccinimidyl tartrate; 3,3′-dithiobis(sulfosuccinimidyl propionate); 3,3′-dithiobis(sulfosuccinimidyl propionate) 1-ethyl-3-[3-dimethylaminopropyl]carbodiimide hydrochloride; Ethylene Glycol bis(succinimidyl succinate); N-(E-maleimidocaproic acid hydrazide); [N-(E-maleimidocaproyloxy)-succinimide ester]; N-Maleimidobutyryloxysuccinimide ester; Hydroxylamine.HCl; Maleimide-PEG-succinimidyl carboxy methyl; m-Maleimidobenzoyl-N-hydroxysuccinimide Ester; N-Hydroxysuccinimidyl-4-azidosalicylic acid; N-(p-Maleimidophenyl isocyanate); N-Succinimidyl(4-iodoacetyl) Aminobenzoate; Succinimidyl-4-(N-maleimidomethyl)cyclohexane-1-carboxylate; Succinimidyl 4-(p-maleimidophenyl) Butyrate; Sulfo Disulfosuccinimidyl Tartrate; [N-(E-maleimidocaproyloxy)-sulfo succinimide ester; N-Maleimidobutyryloxysulfosuccinimide ester; N-Hydroxysulfosuccinimidyl-4-azidobenzoate; m-Maleimidobenzoyl-N-hydroxysulfosuccinimide Ester; Sulfosuccinimidyl (4-azidophenyl)-1,3 dithio propionate; Sulfosuccinimidyl 2-(m-azido-o-nitrobenzamido)-ethyl-1,3′-dithio propionate; Sulfosuccinimidyl 6-(4′-azido-2′-nitrophenylamino) hexanoate; Sulfosuccinimidyl-2-(p-azidosalicylamido)ethyl-1,3-dithiopropionate; N-(Sulfosuccinimidyl(4-iodoacetyl)Aminobenzoate); Sulfosuccinimidyl-4-(N-maleimidomethyl)cyclohexane-1-carboxylate; Sulfosuccinimidyl 4-(p-maleimidophenyl) Butyrate; and mixtures of these. In some embodiments the cross linking agent is mone- or poly-ethylene glycol diglycidil ether. In some embodiments the cross linker is a homobifunctional cross linking agent such as glutaraldehyde.

As used herein, “denaturing” is the process of modifying the quaternary, tertiary and secondary molecular structure of a protein from its natural, original or native state. For example, such as by breaking weak bonds (e.g., hydrogen bonds), which are responsible for the highly ordered structure of the protein in its natural state. The process can be accomplished by, for example: physical means, such as by heating, sonication or shearing; by chemical means such as acid, alkali, inorganic salts and organic solvents (e.g., alcohols, acetone or chloroform); and by radiation. A denatured protein, such as an enzyme, losses its original biological activity. In some instances, the denaturing process is reversible, such that the protein molecular structure is regained by the re-forming of the original bonding interactions at least to the degree that the original biological function of the protein is restored. In other instances, the denaturing process is irreversible or non-reversible, such that the original and biological function of the protein is not restored. Cross-linking, for example after denaturing, can reduce or eliminate the reversibility of the denaturing process.

The degree of denaturing can be expressed as a percent of protein molecules that have been denatured, such as a mole percent. Some methods of denaturing can be more efficient than others. For example, under some conditions, sonication applied to BSA can denature about 30-40% of the protein and the denaturing is reversible. When BSA is denatured it undergoes two structural stages. The first stage is reversible whilst the second stage is irreversible (e.g., non-reversible) but does not necessarily result in a complete destruction of the ordered structure. For example, heating up to 65° C. can be regarded as the first stage, with subsequent heating above that as the second stage. At higher temperatures, further transformations are seen. In some embodiments, BSA is denatured by heating above about 65° C. (e.g., above about 70° C., above about 80° C., above about 90° C., above about 100° C., above about 110° C., above about 120° C.), below about 200° C. (below about 190° C., 180° C., 170° C., 160° C., 150° C.), and for at least about 1 minute (e.g., at least about 2, 3, 4, 5, 10 or 20 minutes) but less than about 24 hours (e.g., less than about 12, 10, 8, 6, 4, 2 1 hour). Embodiments include any ranges herein described, for example heating above about 90° C. but below about 150° C. and for at least 2 minutes but less than one hour.

In some embodiments the proteinaceous material used in the compositions and structures described herein are at least about 20% to about 100% (e.g., 20%, 30%, 40%, 50%, 60%, 70%, 80%, 90%, or 100%) denatured. In some embodiments, less than 50% of the denatured protein reverts back to its natural state (e.g., less than 40%, less than 30%, less than 20%, less than 10%, less than 1%). Therefore, the reversibility of the denaturing can be described as being 50% reversible, 40% reversible (60% irreversible), 30% reversible (70% irreversible), 20% reversible (80% irreversible), 10% reversible (90% irreversible) or even 0% reversible (100% irreversible).

As used herein “carbon nanotubes” and “graphene” are allotropes of carbon with sp² carbon atoms arranged in a hexagonal, honeycomb lattice. Single layer graphene is a two-dimensional material, and is a single layer of graphite. As used herein, more than one layer of graphene can be referred to as graphene, for example between 1 and 200 layers (e.g., about 1 to 100 layers, about 1 to 50 layers, about 1 to 10 layers). Carbon nanotubes are hollow, cylindrical structures, formed as a sheet of graphene rolled into a cylinder. The allotropes of carbon can include some functionalization, such as oxygen, carboxylates, epoxides, amines, amides and combinations of these, as described below.

Graphene can be produced is high purity using chemical vapor deposition on clean metal surfaces and through exfoliation of pure graphite. The exfoliation method of graphite includes using an adhesive which is pressed on the graphite surface repeatedly until a few or even one layer is obtained. These methods can be laborious and impractical, although they can produce graphene that is pure (e.g., greater than 99 wt. % carbon). As will be described below, reduced graphene oxide (rGO) can be used in many applications where graphene is useful since it has similar electrical, chemical and mechanical properties. Reduced graphene also has some advantages, such as chemically reactive oxygen based groups that can be exploited for further chemical transformations. In addition, rGO can be prepared more efficiently. In any case, both pure graphene and reduced graphene oxide can be used in embodiments for making e.Blocker and coated electrodes.

An efficient process for forming graphene oxide is the exfoliation of graphite oxide. As used herein “graphene oxide” is a material that can be formed from the oxidation of graphene or exfoliation of graphite oxide. In a first step for producing graphene oxide, graphite is oxidized. Several methods for oxidation are known, one common method known as the Hummers and Offeman method, in which graphite is treated with a mixture of sulphuric acid, sodium nitrate and potassium permanganate (a very strong oxidizer). Other methods are known to be more efficient, reaching levels of 70% oxidisation, by using increased quantities of potassium permanganate, and adding phosphoric acid combined with the sulphuric acid, instead of adding sodium nitrate. Exfoliation of graphene oxide provides graphite oxide and can be done by several methods. Sonication can be a very time-efficient way of exfoliating graphite oxide, and it is extremely successful at exfoliating graphene (almost to levels of full exfoliation), but it can also heavily damage the graphene flakes, reducing them in surface size from microns to nanometres, and also produces a wide variety of graphene platelet sizes. Mechanically stirring is a much less destructive approach, but can take much longer to accomplish.

Graphite oxide and graphene oxide are very similar, chemically, but structurally, they are very different. Both are compounds having carbon, oxygen and hydrogen in variable ratios. In the most oxidized state the oxygen amount can be as high as about 60 wt %. the amount of hydrogen varies depending on the functionalization, for example, the number of epoxy bridges, hydroxyl groups and carboxyl groups. The main difference between graphite oxide and graphene oxide is the interplanar spacing between the individual atomic layers of the compounds, caused by water intercalation. This increased spacing, caused by the oxidisation process, also disrupts the sp² bonding network, meaning that both graphite oxide and graphene oxide are often described as electrical insulators.

Reduced graphene oxide (rGO) is prepared from reduction of graphene oxide by thermal, chemical or electrical treatments. For example, treating the graphene oxide with; hydrazine, hydrogen plasma, heating in water, high temperature heating (e.g., under nitrogen/argon) and electrochemical reduction. Whereas graphene can be a single carbon layer ideally comprising only carbon, reduced graphene oxide is similar but contains some degree of oxygen functionalization. The amount of oxygen depends on the degree of reduction and in some materials can vary between about 50 wt % and about 1 wt. % (e.g., between about 30 wt. % and about 5 wt. %).

Reduced graphene oxide can be functionalized or include functional groups. For example, reduced graphene oxide often includes oxygen in the form of carboxyl groups and hydroxyl groups. In some forms, the carboxyl and hydroxyl groups populate the edges of the rGO sheets. As used herein, carbonylated reduced graphene oxide can refer to reduced graphene oxide having carboxyl groups. In some embodiments the amount of oxygen attributable to the carboxyl groups is between about 30 wt. % and about 0.1 wt. % (e.g., between about 10 wt. % and about 1 wt. %). Other forms of functionalization are possible. For example, amine functionaized rGO can be formed by a modified Buchere reaction, wherein ammonia an graphene oxide are reacted using a catalyst such as sodium bisulfite, or epoxide groups on graphene oxide can be opened with p-phenylenediamine. In some embodiments, the amount of nitrogen is between about 30 wt. % and 0.1 wt. % (e.g., between about 10 wt. % and 1 wt. %).

The tube-shaped carbon nanotubes have diameters in the nanometer scale, such as, for example, between about 0.2 and about 20 nm, preferably between about 0.5 and about 10 nm, and more preferably still between about 1 and about 5 nm. These can be single walled carbon nanotubes (SWCNT), multi walled carbon nanotubes (MWCNT) (e.g., a collection of 2 or more nested tubes of continuously increasing diameters, or mixtures of these). The diameters of MWCNT can be larger than the SWCNT, such as between about 1 and about 100 nm (e.g., between about 1 and about 50 nm, between about 10 and 20 nm, between 5 and 15 nm, between about 30 and 50 nm). Depending on how the precursor graphene sheet is rolled up to make a seamless cylinder that is the carbon nanotube, different isomers of carbon nanotube can be made, for example designated as armchair configuration, chiral configuration, and zigzag configuration.

The carbon nanotubes and reduced graphene oxide can include intercalated materials, such as ions and molecules. In some embodiments the carbon nanotubes can be functionalized for example by oxidation to form carboxylic acid groups on the surface, providing CNTs. In addition, in some embodiments, the carbon nanotubes and rGO can be further modified through condensation reactions with the carboxylic acid groups present on the CNTs or rGO (e.g., with alcohols and amines), electrostatic interactions with the carboxylic acid groups (e.g., calcium mediated coupling, or quaternary amines, protonated amine-carboxylate interaction, through cationic polymers or surfactants) or hydrogen bonding through the carboxylic acid groups (e.g., with fatty acids, and other hydrogen bonding molecules). The functionalization can be partial (e.g., wherein less than 90%, less than 80%, less than 60%, less than 50%, less than 40%, less than 30%, less than 20%, less than 10%, more than 10%, more than 20%, more than 30%, more than 40%, more than 50%, more than 60%, more than 70%, more than 80%, of the available carboxylic acid groups are functionalized) or complete, such as functionalizing substantially all the carboxylic acids (e.g., more than 90%, more than 95%,more than 99% of available carboxylic acid groups). In some embodiments the functionalization can be with a redox active compound or fragment (e.g., a metallocene, a viologen), antibody, a DNA strand, an RNA strand, a peptide, an antibody, an enzyme, a molecular receptor, a fragment of one of these or combination of these.

The allotropes of carbon having hexagonal lattices of carbon atoms, such as CNTs and rGO, can confer electroactivity (e.g., conductivity) to the compositions and structures herein described. Other conductive elements such as pure graphene, fullerenes, conductive and semi-conductive particles, rods, fibers and nano-particles (e.g., Gold), and conductive polymers (e.g., polypyrrole, polythiophene, polyaniline) can also be used to replace the CNTs and rGO or blended/combined with CNTs to modulate (e.g., improve) the conductivity, improve the stability and/or improve the stability of the coatings.

Interestingly, some of the embodiments described herein show anisotropy in conductivity. In some embodiments the coatings conduct in a direction perpendicular to the surface of an electrode, equivalent herein to “vertically”, to a greater degree than in directions parallel or tangential to the surface of the electrode, equivalent herein to “laterally”. In Cartesian coordinates this can correspond to higher conduction in the z direction (perpendicular to the electrode surface) than in the x and y directions (e.g., combinations of x and y pointing vectors). For example, the conductivity in the vertical direction is at least two times (e.g. at least 3 times, 4 times, 5 times, 10 times, 100 times, 1000 times) higher than that in the lateral direction.

As used herein the term “comprising” or “comprises” is used in reference to compositions, methods, and respective component(s) thereof, that are essential to the claimed invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the claimed invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±5% (e.g., ±4%, ±3%, ±2%, ±1%) of the value being referred to.

Where a range of values is provided, each numerical value between the upper and lower limits of the range is contemplated and disclosed herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that can be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments of the various aspects described herein can be illustrated by the following numbered paragraphs.

1. A composition comprising a mixture of an allotrope of carbon having carbon atoms arranged in a hexagonal lattice and a proteinaceous material, wherein the proteinaceous material is non-reversibly denatured.

2. The composition of paragraph 1, wherein the allotrope of carbon is a functionalized material.

3. The composition of paragraph 1 or 2, wherein the allotrope of carbon is carbon nanotubes, reduced graphene oxide or mixtures thereof.

4. The composition of paragraph 3, wherein the carbon nanotube is carboxylated carbon nanotubes (CNTs) or aminated carbon nanotubes.

5. The composition of paragraph 3, wherein the reduced graphene oxide is a carboxylated reduced graphene oxide or an aminated reduced graphene oxide.

6. The composition of any one of paragraphs 1-5, wherein the proteinaceous material is cross-linked.

7. The composition of any one of paragraphs 1-6, wherein the proteinaceous material is bovine serum albumin (BSA).

8. The composition of any one of paragraphs 1-7, wherein the mixture further comprises a capture agent.

9. The composition of any one of paragraphs 1-8, further comprising a conductive surface.

10. An electrode comprising:
a conductive surface; and
a mixture of an allotrope of carbon having carbon atoms arranged in a hexagonal lattice and a proteinaceous material coated on at least a part of said conductive surface, and
wherein the proteinaceous material is non-reversibly denatured.

11. The electrode of paragraph 10, wherein the allotrope of carbon is a functionalized material.

12. The electrode of paragraph 10, wherein the allotrope of carbon is carbon nanotubes, reduced graphene oxide or mixtures thereof.

13. The electrode of paragraph 12, wherein the carbon nanotube is carboxylated carbon nanotubes (CNTs) or aminated carbon nanotubes.

14. The electrode of paragraph 12, wherein the reduced graphene oxide is carboxylated reduced graphene oxide or aminated reduced graphene oxide.

15. The electrode of any one of paragraphs 10-14, wherein the proteinaceous material is cross-linked.

16. The electrode of any one of paragraphs 10-15, wherein the proteinaceous material is BSA.

17. The electrode of any one of paragraphs 10-16, wherein the mixture further comprises a capture agent.

18. The electrode of any one of paragraphs 10-17, wherein the mixture conducts vertically to a greater degree than laterally.

19. The electrode of any one of paragraphs 10-18, wherein the electrode is multiplexed.

20. A method of making an electrode coating composition, the method comprising:
mixing an allotrope of carbon having carbon atoms arranged in a hexagonal lattice and proteinaceous material in a solution, wherein the proteinaceous material is non-reversibly denatured prior to or after mixing with the carbon allotrope.

21. The method of paragraph 20, wherein the allotrope of carbon is a functionalized material.

22. The method of paragraph 20, wherein the allotrope of carbon is carbon nanotubes, reduced graphene oxide or mixtures thereof.

23. The method of paragraph 22, wherein the carbon nanotube is carboxylated carbon nanotubes (CNTs) or aminated carbon nanotubes.

24. The method of paragraph 22, wherein the reduced graphene oxide is carboxylated reduced graphene oxide or aminated reduced graphene oxide.

25. The method of any one of paragraphs 20-24, further comprising sonicating the allotrope of carbon and proteinaceous mixture.

26. The method of any one of paragraphs 20-25, wherein the proteinaceous material is denatured by application of heat.

27. The method of any one of paragraphs 20-26, further comprising cross linking the proteinaceous material

28. The method of any one of paragraphs 20-27, wherein the proteinaceous material is BSA.

29. The method of any one of paragraphs 20-28, further comprising purifying the allotrope of carbon and proteinaceous mixture.

30. The method of any one of paragraphs 20-29, wherein the solution is an aqueous solution.

31. A method of making a coated electrode, the method comprising;
coating at least a portion of a conducting surface with a mixture of an allotrope of carbon having atoms arranged in a hexagonal lattice and a proteinaceous material, wherein the proteinaceous material is non-reversibly denatured.

32. The method of paragraph 31, wherein the allotrope of carbon is a functionalized material.

33. The method of paragraph 31, wherein the allotrope of carbon is carbon nanotubes, reduced graphene oxide or mixtures thereof.

34. The method of paragraph 33, wherein the carbon nanotube is carboxylated carbon nanotubes (CNTs) or aminated carbon nanotubes.

35. The method of paragraph 33, wherein the reduced graphene oxide is carboxylated reduced graphene oxide or aminated reduced graphene oxide.

36. The method of any one of paragraphs 31-35, further comprising cross linking the proteinaceous material.

37. The method of any one of paragraphs 31-36, wherein the proteinaceous material is BSA.

38. The method of any one of paragraphs 3136, wherein the mixture further comprises a capture agent.

### Examples

e.Block with Carbon Nanotubes
Preparation of e.Block with Carbon Nanotubes

Carboxylated carbon nanotubes (1.7 mg) and 5 mg of BSA were mixed in 1 mL phosphate buffer saline solution (PBS). The solution was subsequently homogenized by sonication in a probe sonicator (125 watts and 20 KHz) at 50% amplitude for 30 minutes at room temperature. A thermal denaturation step at 105° C. for 5 minutes followed and subsequently the sonication step was repeated in order to further homogenize the mixture. CNTs aggregates were separated by centrifugation at a relative centrifugal force of 16.1 g for 15 minutes. The supernatant containing the e.Blocker was separated and kept for further use, while the sedimented CNTs were discarded.

In some optional embodiments, the BSA can be denatured in a first step, for example by heating as described above. Subsequently, CNTs can be added to the solution and homogenized.

In both these optional embodiments, the CNTs can be functionalized with a chemical group or a molecular receptor (e.g. Antibody, DNA strand) covalently linked to the CNTs.

To test the effects of denaturing, conditions as described in U.S. Pat. No. 8,778,269, herein incorporated by reference, were used to make a blocking agent. To this end, a mixture of BSA (5 mg/ml) and carboxylic functionalized single-walled carbon nanotubes (0.1 mg/ml) in PBS was made and sonicated in a probe sonicator (125 watts and 20 KHz) at 50% amplitude for 30 minutes at room temperature. Therefore, the heat denaturation step used for the preparation of e.Blocker, was not used in this example. The mixture was subsequently centrifuged at a relative centrifugal force of 16.1 g for 15 minutes. The supernatant was collected (referred to as “PTNTM”) and kept for further use while the sediment was discarded.

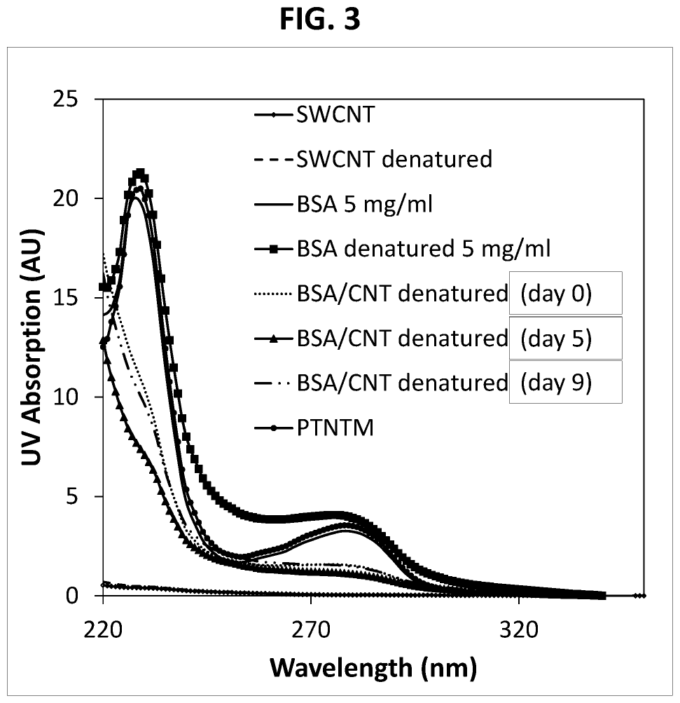

The absorption of the e.Block in the region of UV light shows reproducible spectra across different batches. FIG. 3 shows there is a slight drop of the bands at 230 nm and 280 nm (dotted-line for BSA/CNTs denatured day 0, triangle-line for BSA/CNTs day 5, and dash-dot-dot-line for BSA/CNTs denatured day 9) which is indicative of the denaturation of the BSA. This specific transformation, which suggests random coil distribution of the protein is not observed in original BSA (solid black line), BSA denatured (Square-marker-line) or PTNTM (circle-marked-line), all which show a peak around 230 and 280 nm. These results indicate that both the contribution of CNTs and a denaturation step are hugely beneficial to the formulation to prepare the e.Block. SWCNT (diamond-marker-line) and SWCNT denatured (dashed-line) show almost no adsorption in the scanned region.

Coating of Sensors with e.Block

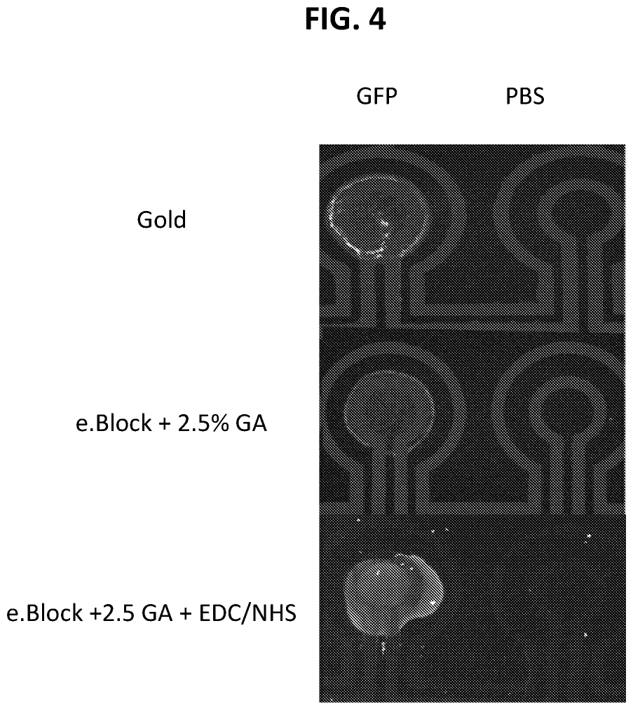

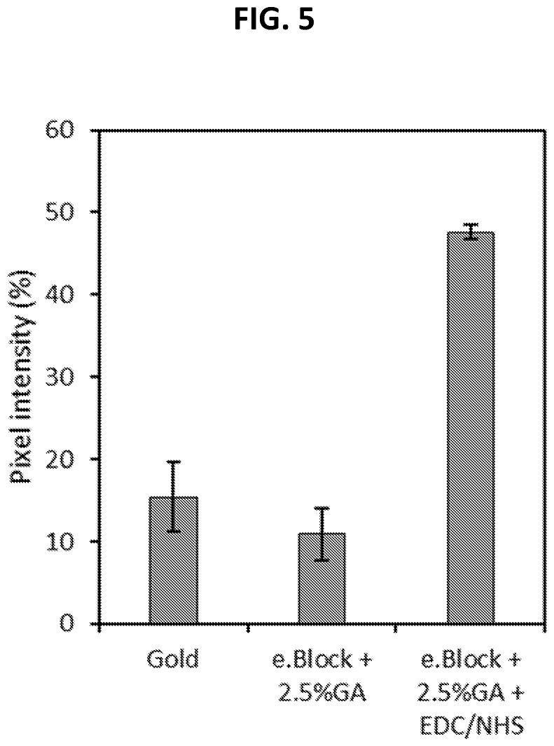

Prior to coating of an electrode's surface, e.Block was mixed with glutaraldehyde (GA) to a final concentration of 2.5% of and the mixture was immediately drop-casted onto electrochemical sensors. The combination was then incubated in a water saturated atmosphere for a period of 24 hours before being thoroughly rinsed using PBS. This provides a coating that is stable, chemically inert and that can be functionalized with a bioreceptor if desired. FIG. 4 is a fluorescence image of an array of 6 gold sensors. The image shows, from top to bottom, an unmodified gold sensor, a gold sensor incubated with e.Block+2.5% glutaraldehyde (GA) for 24 hours and a sensor treated with e.Block, 2.5% GA and (1-ethyl-3-(3-dimethylaminopropyl)carbodiimide hydrochloride)/Dicylohexylcarbodiimide (EDC/NHS) to enable the immobilization of molecular receptor via carbodiimide coupling. The sensors shown under the “GFP” label, were spotted with a drop of 0.46 mg/ml of green fluorescent protein (GFP) and incubated overnight at 4° C. The sensors shown under the “PBS” label were spotted PBS as a negative control. FIG. 5 shows relative fluorescent pixel intensity on the surface of each sensor compared to the PBS control. The sensor modified with e.Block and 2.5% GA shows no significant enhancement in fluorescence compared to the gold electrode, indicating that the reactivity of the e.Block treated with GA was negligible. In contrast to this, the surfaces activated with EDC/NHS prior to spotting showed a strong fluorescent signal, demonstrating the ability to covalently immobilize molecular receptors on the e.Blocker via carbodiimide coupling.

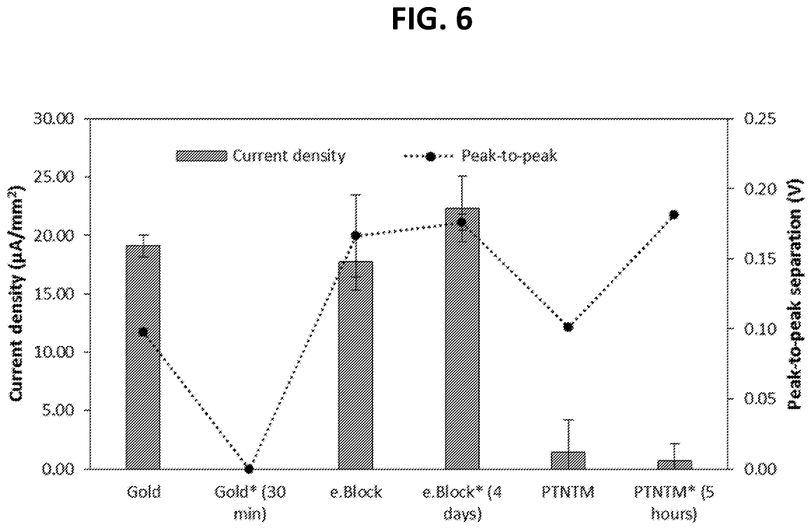

The comparative sample, PTNTM, was also tested on the gold sensors. PTNTM was drop-casted on the surface of gold sensors and incubated in a water saturated atmosphere for a period of 24 hours before being thoroughly rinsed using PBS. Cross linking with GA was omitted. Subsequent to this treatment, the coating was electrochemically characterized and showed current in only 1 out of 4 electrodes tested. The comparative of oxidation current density (bars) and peak to peak distance (filled circle markers) of a 5 mM ferri/ferrocyanide results of non-treated, e.Block treated and PTNTM treated are shown in FIG. 6 in a solution containing 1% BSA. As shown, non-treated gold is quickly passivated (by 30 min); e.Block shows high current density with no significant change over 4 days; while PTNTM shows low initial current that is unchanged over 5 hours.

### Sterilization

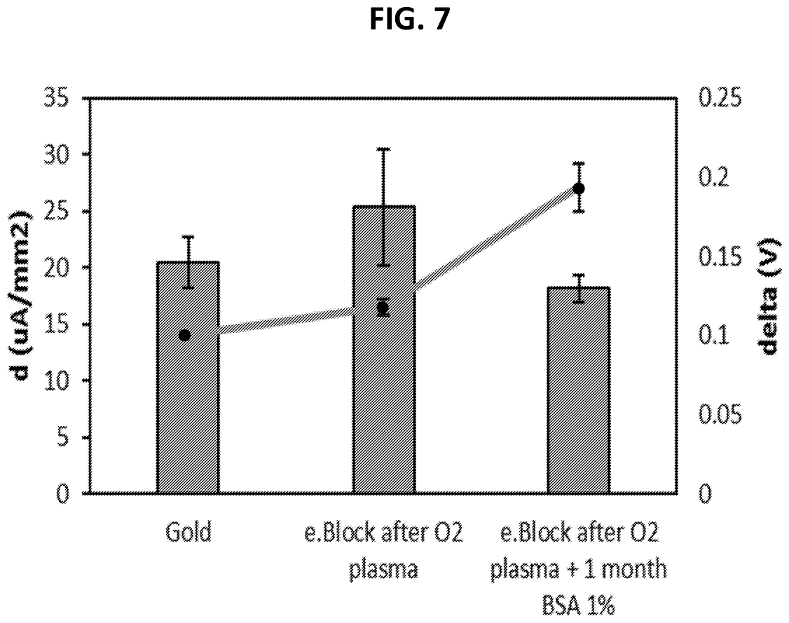

Sensors that are coated with e.Block can be treated with oxygen plasma (0.3 mbar, 50 watt, 4 minutes) and maintain their activity for at least one month. FIG. 7. Shows the electrochemical signal from the oxidation current density and peak to peak distance of a 5 mM ferri/ferrocyanide in PBS buffer solution measured at bare gold electrodes, electrodes modified with e.Block and treated with a O₂ plasma (0.3 mbar, 50 watt, 4 minutes), and the signal of these electrodes after incubation in 1% BSA for 1 month. This can be useful, for example, for surface sterilization prior cell seeding.

### Functionalization

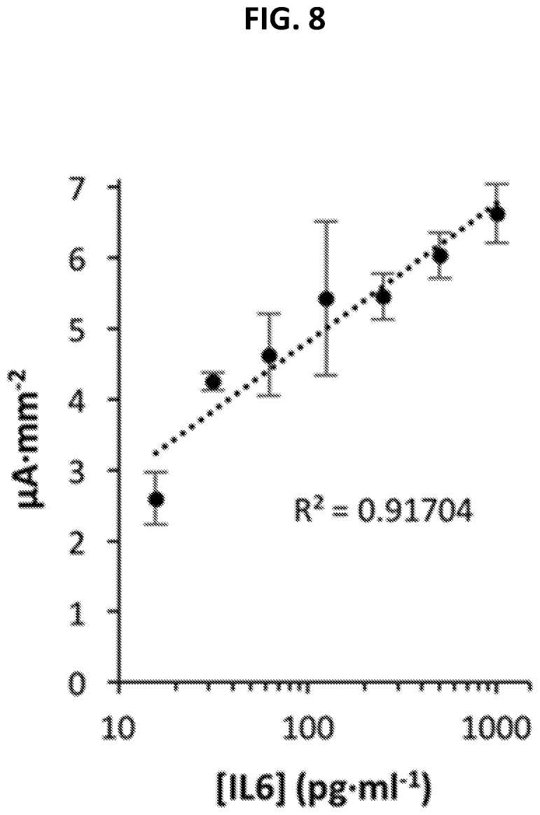

Electrodes coated with e.Block can be functionalized via EDC/NHS coupling chemistry without compromising the stability of the coating. The e.Block coated sensors were functionalized with capturing anti-IL6 (FIG. 1) and were able to quantify the presence of IL6 in a matrix containing 1% BSA with high sensitivity. FIG. 8 is a plot showing the electrochemical signals from the oxidation of precipitated TMB recorded for varying concentration of IL6 in the presence of 1% BSA. The detection range spans at least three orders of magnitude, from at least 10 pg/mL to 1000 pg/mL.

Without e.Block, diffusion of electrochemically active compounds from specific electrodes would accumulate on neighbor control electrodes. The antifouling properties of e.Block allow a reduction of signal in control sensors and therefore an improvement reduction in the limit of detection. Due to the good antifouling properties, antibody functionalized e.Block modified sensors can be conveniently prepared and stored in 1% BSA for at least 1 week preserving the electrochemical activity and sensitivity. This is also particularly relevant to stabilize the immobilize receptor and extend the sensor shelf life while retaining electroactivity. Also, complete regeneration of the antibody functionalized e.Block surface is possible by simply flushing the electrodes with 10 mM HCl glycine, as described below.

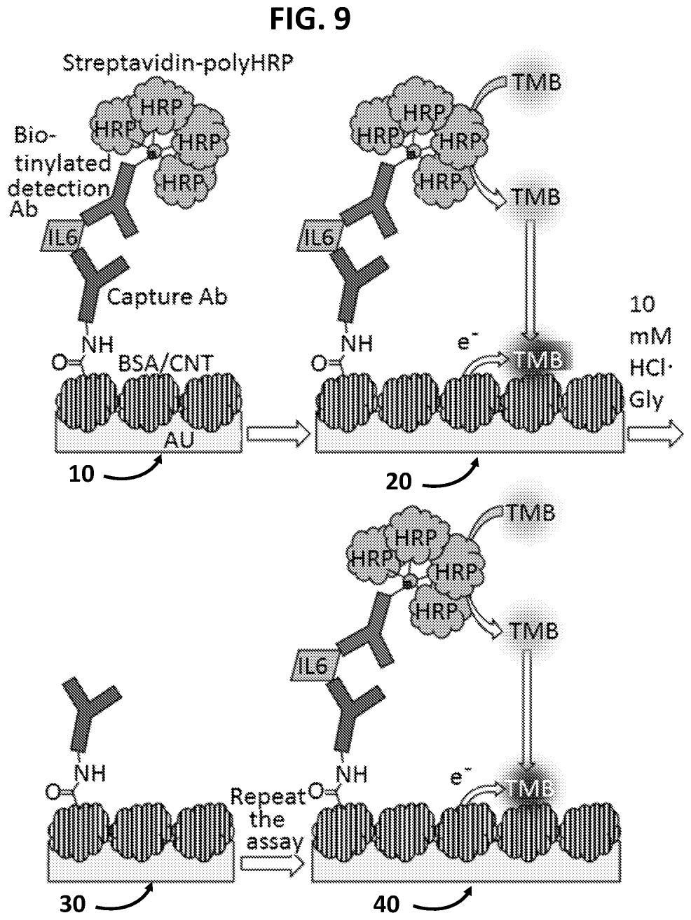

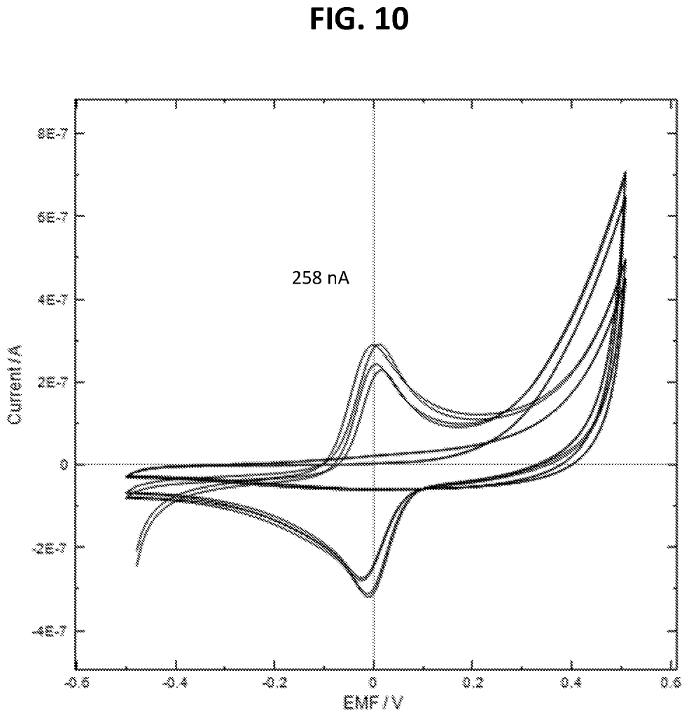

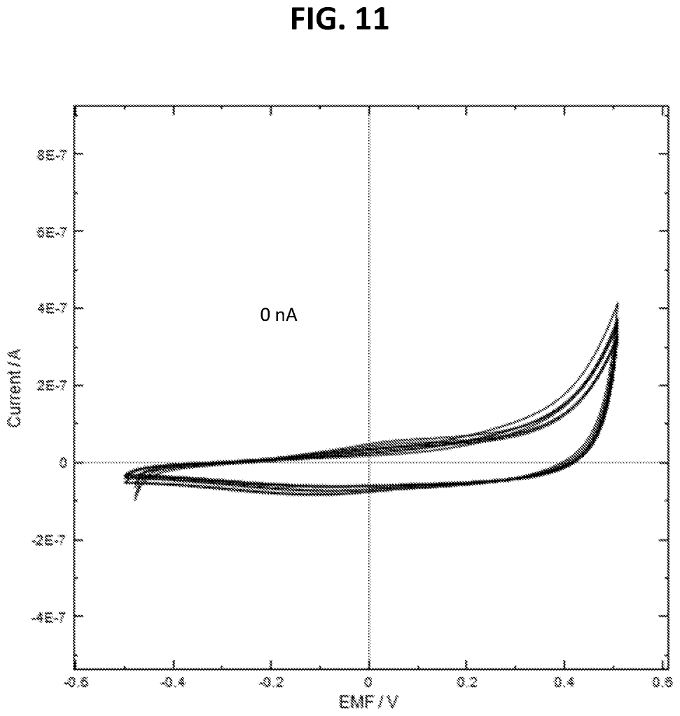

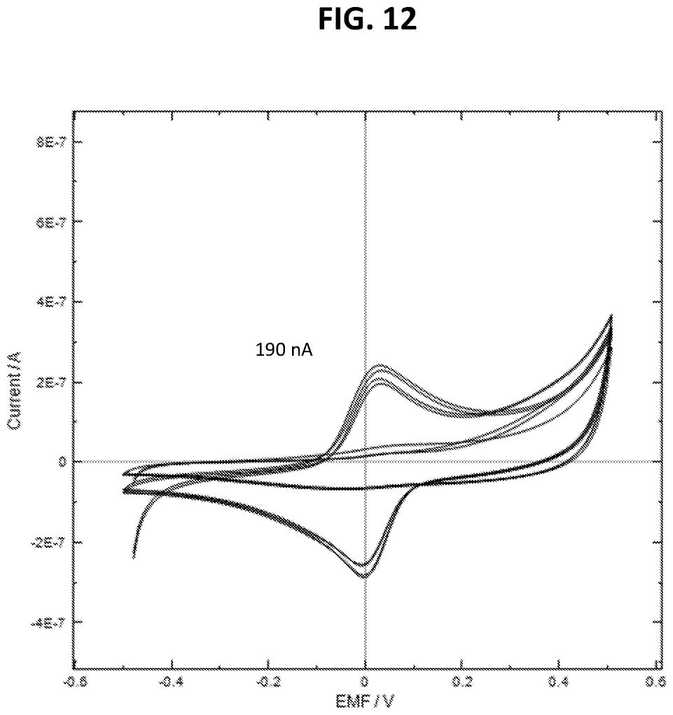

FIG. 9 is a diagrammatic depiction showing the performance of a gold electrode surface functionalized with capture anti-IL6, stored for a week in 1% BSA, and then used to carry out the detection of 200 pg/mL of IL6 in a matrix containing 1% BSA. The figure shows the electrode in four different states: state 10 shows the electrode with captured IL6 and detection antibody, state 20 shows the electrode precipitating and electrochemically detecting TMB, state 30 shows the electrode after being washed with 10 mM HCl·Gly (wherein the capture antibody, TMB and IL6 have been washed away), state 40 shows the electrode being used again to detect IL6 using detection antibody and TMB. FIGS. 10, 11 and 12 are voltammograms created using the electrode in the states depicted in FIG. 9. The voltammogram depicted in FIG. 10 shows the redox peaks of precipitated TMB after an IL6 detection assay depicted by 20 (FIG. 9) where the peak current is 258 nA. Pure TMB presents two very clear reversible redox peaks. Regeneration of the surface gives rise to the voltammogram depicted by FIG. 11, corresponding to electrode state 30 (FIG. 9), and has no redox peaks (0 nM above baseline). The repeat assay is shown in the voltammogram depicted by FIG. 12, corresponding to electrode state 40 (FIG. 9). The two very clear redox peaks again correspond to TMB and show that the electrode has been regenerated. The peak current of 190 nA corresponds to 74% of the original signal. These experiments shows that the sensor can be regenerated and reused to detect IL-6 in solution with minimal loss of sensitivity.

e.Block with Reduced Graphene Oxide
Preparation of e.Block with Reduced Graphene Oxide

Amine modified reduced graphene oxide, RG-Amino, (product number 805432) and carboxylated reduced graphene oxide, RG-Carboxylated, (product number 805424) was purchased from Sigma-Aldrich (Milwaukee, Wis.). 1.7 mg of either carboxylated or aminated reduced graphene oxide and 5 mg of BSA were mixed in 1 mL phosphate buffer saline solution (PBS). The solution was subsequently homogenized by sonication in a probe sonicator (125 watts and 20 KHz) at 50% amplitude for 30 minutes at room temperature. A thermal denaturation step at 105° C. for 5 minutes followed. Reduced graphene aggregates were separated by centrifugation at a relative centrifugal force of 16.1 g for 15 minutes. The supernatant containing the e.Block was separated and kept for further use, while the sedimented reduced graphene was discarded.

### Coating of Electrode Surface

The same method used for coating with e.Block made using CNTs can be used for coating an electrode with e.Block made using reduced graphene. Therefore, prior to coating of an electrode's surface e.Block was mixed with glutaraldehyde (GA) to a final concentration of 2.5% and the mixture was immediately drop-casted on electrochemical sensors. The combination was incubated for a period of 24 hours before being thoroughly rinsed using PBS.

Results with Reduced Graphene e.Block

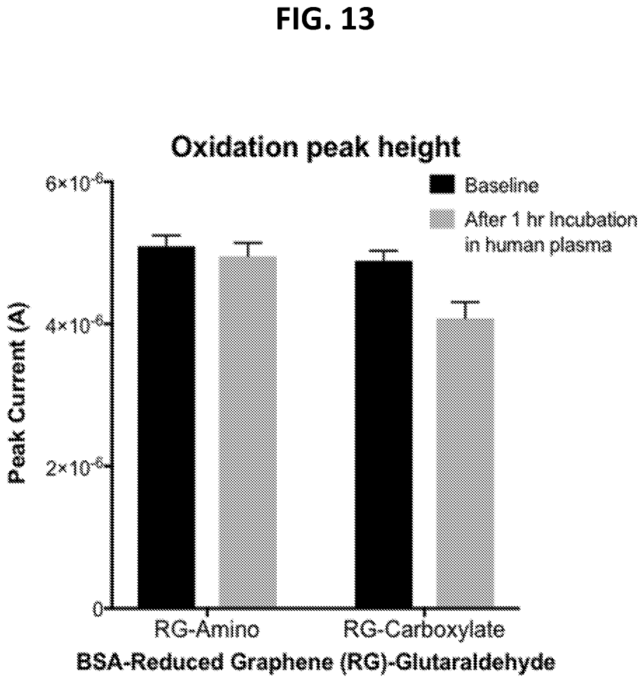

Reduced graphene provides an alternative to CNTs for preparation of e.Block. The e.Block made with two different types of reduced graphene have been exemplified namely; aminated reduced graphene and carboxylated reduced graphene. The electrochemical surfaces modified with e.Blocker was incubated with un-diluted human plasma for 60 minutes. The oxidation peak current of a 5 mM ferri/ferrocyanide in PBS was monitored before and after incubation. FIG. 13 demonstrates that both type of e.Blockers made with reduced graphene exhibit a limited decreased in sensor sensitivity after incubation with human plasma, therefore, retaining most of the electrodes conductivity.

1.-38. (canceled)

39. A composition comprising a mixture of a conducting element and a proteinaceous material, wherein the proteinaceous material is non-reversibly denatured.

40. the composition of claim 39, wherein the proteinaceous material is cross-linked.

41. The composition of claim 39, wherein the proteinaceous material is bovine serum albumin (BSA).

42. The composition of claim 39, wherein the mixture further comprises a capture agent.

43. The composition of claim 39, wherein the conducting element comprises conductive and semi-conductive particles, rods, fibers, nano-particles or polymers.

44. The composition of claim 43, wherein the conducting element comprises gold.

45. The composition of claim 43, wherein the conducting element comprises an allotrope of carbon atoms arranged in a hexagonal lattice.

46. The composition of claim 45, wherein the allotrope of carbon is a functionalized material.

47. The composition of claim 45, wherein the allotrope of carbon is carbon nanotubes, reduced graphene oxide or mixtures thereof

48. The composition of claim 47, wherein the carbon nanotube is carboxylated carbon nanotubes (CNTs) or aminated carbon nanotubes.

49. The composition of claim 47, wherein the reduced graphene oxide is a carboxylated reduced graphene oxide or an aminated reduced graphene oxide.

50. An electrode comprising:
a conductive surface; and
a mixture of a conducting element and a proteinaceous material coated on at least a part of said conductive surface, and wherein the proteinaceous material is non-reversibly denatured.

51. The electrode of claim 50, wherein the proteinaceous material is cross-linked.

52. The electrode of claim 50, wherein the proteinaceous material is BSA.

53. The electrode of claim 50, wherein the mixture further comprises a capture agent.

54. The electrode of claim 50, wherein the mixture conducts vertically to a greater degree than laterally.

55. The electrode of claim 50, wherein the conducting element comprises conductive and semi-conductive particles rods, fibers, nano-particles or polymers.

56. The electrode of claim 55, wherein the conducting element comprises gold.

57. The electrode of claim 55, wherein the conducting element comprises an allotrope of carbon atoms arranged in a hexagonal lattice.

58. The electrode according to claim 50, wherein the electrode is multiplexed.

59. A method of making an electrode coating composition, the method comprising:
mixing a conducting element and proteinaceous material in a solution, wherein the proteinaceous material is non-reversibly denatured prior to or after mixing with the carbon allotrope.
